(19) United States
(12) Patent Application Publication
(10) Pub. No.: US 2010/0274129 A1
(43) Pub. Date: Oct. 28, 2010

(54) Apparatus And Methods for Separating Pericardial Tissue From The Epicardium of the Heart

(76) Inventor: Michael D. Hooven, Cincinnati, OH (US)

Correspondence Address: COOK ALEX LTD, SUITE 2850, 200 WEST ADAMS STREET, CHICAGO, IL, 60606, US

(21) Appl. No.: 12/767,280

(22) Filed: Apr. 26, 2010

Related U.S. Application Data
(60) Provisional application No. 61/172,596, filed on Apr. 24, 2009.

(51) Int. Cl. A61B 17/00; A61B 6/00; A61B 17/34

(52) U.S. Cl. 600/435; 606/190; 606/185

(57) ABSTRACT

Systems and methods for separating pericardial tissue from the epicardium of the heart are disclosed. The apparatus includes a catheter comprising an elongated body, including a proximal end and a distal end and a lumen extending therebetween. The apparatus further comprises a needle carried at the distal end of the catheter. The needle includes a proximal end, a distal end, and a lumen extending between the proximal and distal ends, and is in fluid communication with the catheter lumen. The needle is of a length sufficient to penetrate myocardial tissue of the heart, from the endocardium to and through the epicardium. A coupling on the catheter is provided for communication with a fluid source, to facilitate flow of fluid through the catheter lumen and needle lumen. Fluid can thereby flow through the catheter and needle lumens to a location between the epicardium and pericardial tissue. Fluid flow to this location serves, among other things, to separate the pericardial tissue from the epicardium of the heart.

### CROSS REFERENCE TO RELATED APPLICATION

This application claims the benefit of the filing date of U.S. Provisional Application Ser. No. 61/172,596, filed Apr. 24, 2009, the entire contents thereof being incorporated by reference.

### FIELD OF THE INVENTION

The present application relates to a method and apparatus for separating pericardial tissue from the epicardium of the heart.

### BACKGROUND

Obtaining access to certain surfaces of the heart is often desired and/or necessary to perform various surgical procedures of the heart and of the area surrounding the heart. Surgical procedures involving the heart and cardiac tissue may be carried out in an open surgical procedure, where the breastbone is divided and the surgeon has direct access to the heart. Alternatively, and usually more preferably, however, surgical procedures involving the heart are performed through a minimally invasive route. This may include, for example, accessing certain areas of the heart through a catheter that has been inserted through the vascular system of a patient to a location in the interior of the heart. Surgical or diagnostic instruments inserted through the catheter may then be manipulated within the interior of the heart to thereby gain access to a desired surgical site.

Other minimally invasive approaches to the heart include access between the ribs (“intercostal”) or below the sternum (“subxyphoid”). These approaches are typically employed for accessing the heart via the outer surface or epicardium. Accessing the epicardial surface of the heart typically requires separation of the pericardium from the epicardial surface, as is well known in the field.

The pericardium is a membranous sac that encloses the heart. It consists of an outer layer of dense fibrous tissue and an inner serous layer, termed the epicardium, which directly surrounds the heart. Throughout the description and claims that follow, the phrase “within the pericardium” or “within the pericardial space” is used to mean any of the body tissue or fluid found inside of the dense outer layer of the pericardium, including the outer surface of the heart, but not including the interior of the heart.

By way of the method and system of the present disclosure, pericardial effusion is utilized to obtain direct percutaneous access to the pericardial space. This is because, in the absence of a pericardial effusion, any attempt to introduce a sharp object percutaneously through the pericardium could result in damaging the myocardium.

To reduce the likelihood of this occurring, the method and system of the present disclosure includes means for distending the pericardium from the heart by injecting a small volume of fluid into the pericardium from the interior of the heart, thus creating a pericardial effusion. This injection extends the pericardium away from the heart. A conventional needle having a lumen therethrough may then be inserted from the desired percutaneous location into the body tissue until a tip thereof punctures the distended pericardium at a selected location to provide the desired access to the heart.

### BRIEF DESCRIPTION OF THE DRAWINGS

FIG. 1 is a view of a human chest, illustrating apparatus and method described herein in an exemplary use, being inserted into a patient's vascular system into the right ventricle of the heart.

FIG. 2 is an enlarged view illustrating apparatus employing a needle, carried by the distal end of a catheter, penetrating the endocardium and myocardium of the right ventricle of the heart.

FIG. 3 illustrates the apparatus of FIG. 2 advanced through the epicardium and delivering fluid to a location between the epicardium and pericardial tissue.

FIG. 4 is a side view of one embodiment of the apparatus for delivering fluid to a location between the epicardium and pericardium of the heart, employing a needle carried by the distal end of a catheter in communication with both a fluid source and an EKG.

FIG. 5 is an enlarged cross-sectional view of the apparatus illustrated in FIG. 4, wherein a fluid pressure monitor and associated controller are in communication with the internal catheter and needle lumen.

FIG. 6A is an enlarged side view of the needle employed in the apparatus of FIG. 2, having a generally helical shape.

FIG. 6B is perspective view of the helical needle of FIG. 6A.

FIG. 6C is a distal end view of the helical needle of FIGS. 6A and 6B.

FIG. 6D is a proximal end view of the helical needle of FIGS. 6A and 6B.

FIG. 7A is a side view of apparatus that may be used as illustrated in FIG. 1, employing a catheter including a proximal end and a distal end.

FIG. 7B is a side cross sectional view of the catheter illustrated in FIG. 7A, having an internal lumen between the proximal and distal ends and a helical needle carried by and retracted into the distal end of the catheter sheath.

FIG. 7C is an enlarged side cross sectional view of the distal end of the catheter illustrated in FIG. 7B.

FIG. 7D is an end view of the distal end of the catheter illustrated in FIGS. 7A and 7B.

FIG. 8A is a side view of the apparatus illustrated in FIG. 7, employing a catheter including a proximal end and a distal end, with the helical needle in an extended position.

FIG. 8B is a side cross sectional view of the catheter illustrated in FIG. 8A, having an internal lumen between the proximal and distal ends and a helical needle carried by and extended out of the distal end of the catheter sheath.

FIG. 8C is an enlarged, side cross sectional view of the distal end of the catheter illustrated in FIG. 8B, with a helical needle extending out of the catheter sheath.

FIG. 8D is an end view of the distal end of the catheter illustrated in FIGS. 8A and 8B.

FIG. 8E is an enlarged cross sectional view of the distal end of the catheter illustrated in 8B with an annular fluid channel and a sealed tube having a lumen extending therethrough for introduction of a stylet or guide wire

### SUMMARY

In accordance with one aspect of the present application, an apparatus for separating pericardial tissue from the epicardium of the heart is provided. The apparatus includes a catheter comprising an elongated body, including a proximal end and a distal end and a lumen extending therebetween. The apparatus further comprises a needle carried at the distal end of the catheter. The needle includes a proximal end, a distal end, and a lumen extending between the proximal and distal ends, and is in fluid communication with the catheter lumen. The needle is of a length sufficient to penetrate myocardial tissue of the heart, from the endocardium to and through the epicardium. A coupling on the catheter is provided for communication with a fluid source, to facilitate flow of fluid through the catheter lumen and needle lumen. Fluid can thereby flow through the catheter and needle lumens to a location between the epicardium and pericardial tissue. Fluid flow to this location serves, among other things, to separate the pericardial tissue from the epicardium of the heart.

In accordance with another aspect of the present invention, a method for separating pericardial tissue from the epicardium of the heart is provided. The method preferably includes inserting a needle into the endocardium of the heart and penetrating the myocardium of the heart. The myocardium is penetrated by the needle until a distal end of the needle extends beyond the epicardium, but not penetrating pericardial tissue. The method further comprises delivering fluid through the needle at a pressure sufficient to cause separation of the pericardial tissue from the epicardium of the heart. The method further comprises applying a fluid pressure during penetration of the needle such that fluid is ejected from the needle tip immediately upon exiting the myocardium.

### DETAILED DESCRIPTION

The exemplary embodiments of the present disclosure are described and illustrated below to encompass various methods and apparatus for separating the pericardium from the epicardium. Of course, it will be apparent to those of ordinary skill in the art that the preferred embodiments discussed below are exemplary in nature and may be reconfigured without departing from the scope and spirit of the present disclosure. However, for clarity and precision, the exemplary embodiments as discussed below may include steps, methods, and features that one of ordinary skill should recognize as not being a requisite to fall within the scope of the present disclosure. Hereinafter, the exemplary embodiments of the present disclosure will be described in detail with reference to the drawings.

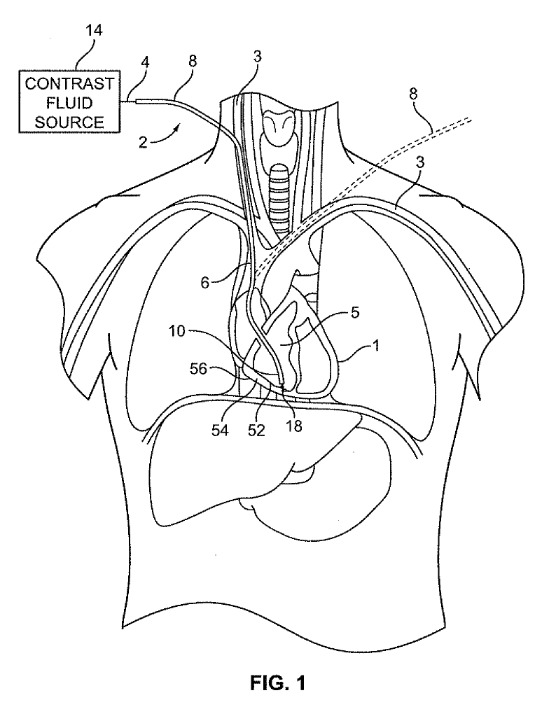

Turning now to a more detailed description, FIG. 1 generally illustrates an embodiment of the claimed apparatus 2 and method in an exemplary use within a patient's body. In use, a guide wire 4 may first be inserted through the vascular system of a patient into the interior of the heart. In one example, the guide wire may be inserted through an artery or vein 3 located in a patient's neck or shoulder, such as the external jugular vein or the subclavian vein as seen in FIG. 1. It is also possible to insert the wire through one or more other arteries or veins that comprise the patient's vascular system to gain access to the interior of the heart. After the guide wire 4 has been inserted into the external jugular vein 3, it is preferably advanced through the superior vena cava to the right atrium, through the tricuspid valve and into the right ventricle 5 of the heart, as seen in FIG. 1.

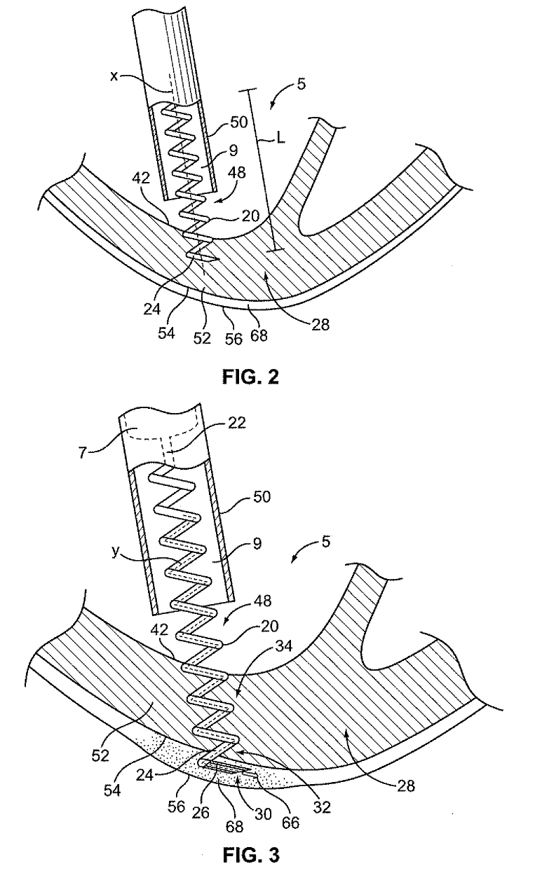

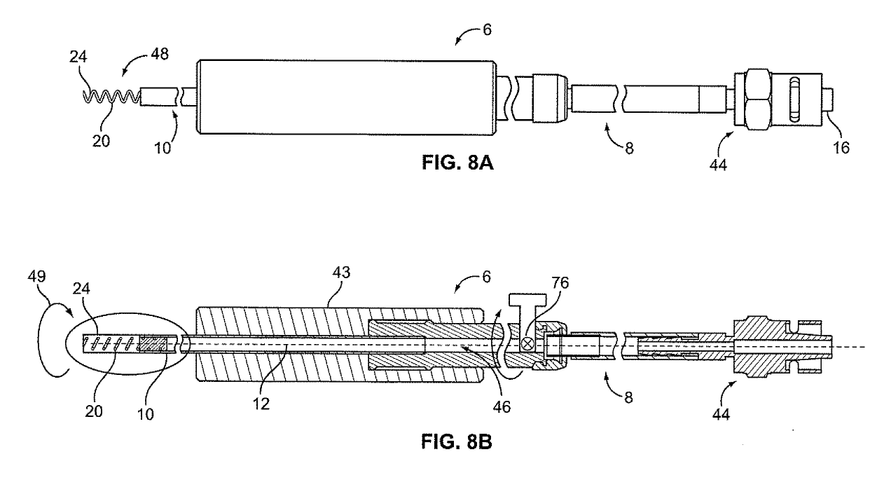

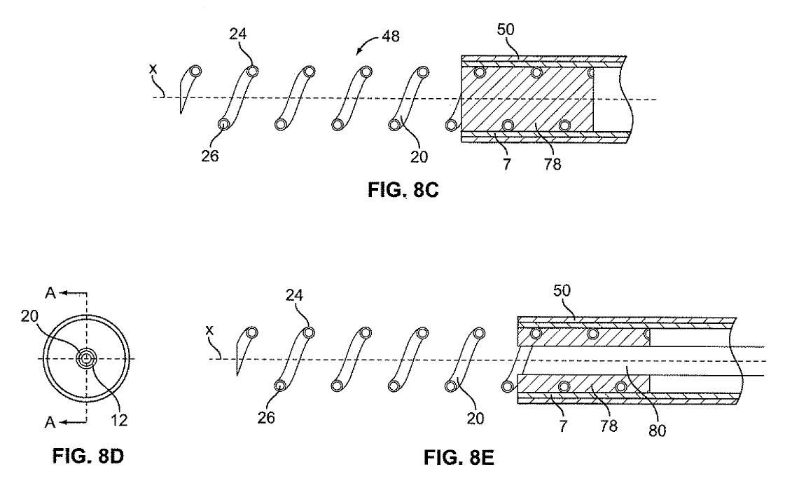

After the guide wire 4 has been inserted into the right ventricle 5, a catheter assembly 6 is advanced over the guide wire into the right ventricle 5, as seen in FIG. 1. This catheter assembly can include either the helical screw catheter designed for guide wire introduction (as shown in FIG. 8E), or it could be a steerable sheath (such as steerable sheath 50, as seen in FIG. 2) as is well known in the art. It may be preferable to locate the distal end of the catheter assembly 6 in the right ventricle 5 for various reasons. For example, the right ventricle is relatively easy to access in relation to other chambers of the heart, while further providing direct tactile feedback to a physician who may be manipulating and/or rotating the catheter assembly 6 or other instruments within the heart, as will be described in further detail below. However, the distal end of the catheter may alternatively be located in one of the other chambers of the heart (the left ventricle and the left and right atriums).

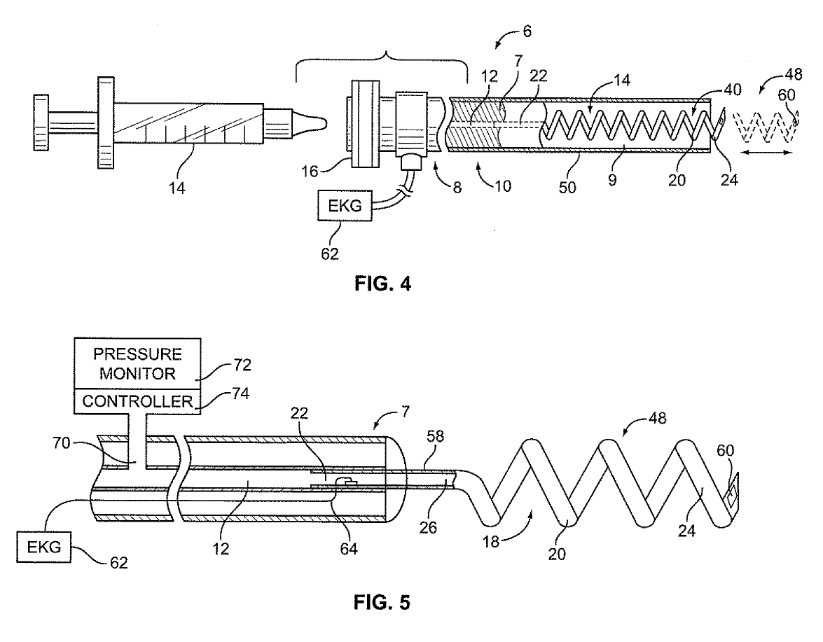

As shown generally in FIG. 1 and in detail in FIGS. 4, 7A and 8A, the catheter assembly 6 preferably comprises an elongated body, having a proximal end 8 and a distal end 10. The catheter assembly 6 may employ a single tubular member or a plurality of relatively movable tubular members, e.g., coaxially disposed one within another. FIG. 4 shows a catheter assembly 6 having an outer sheath 50 and internal tubular member 7 slidably disposed within lumen 9 of the sheath 50. A source of fluid 14, such as a contrast agent or the like, is in communication with the proximal end 8 of the catheter assembly 6. The fluid source 14, shown generically in FIG. 1, may be one or more of various known containers of fluid, including a bottle or bag, a syringe, or pump, or any other source adapted to be placed into fluid communication with the catheter lumen 12. In the embodiment illustrated in FIG. 4, the source of fluid 14 comprises a syringe, for injecting fluid into a luer adapter 16 located at the proximal end 8 of the catheter lumen 12. The syringe may contain up to 20 cc or more of fluid for injection into the catheter lumen 12. The quantity is preferably less than an amount of fluid which, if injected around the heart, would cause an undue tamponade effect.

A penetrating structure 18 is carried by the distal end 10 of the catheter assembly body as illustrated in exemplary FIGS. 1 4, and 7B. In a preferred embodiment, the penetrating structure 18 comprises a needle 20. As seen in further detail in FIG. 5, the needle has a proximal end 22 and a distal end 24, with an interior lumen 26 extending between the needle ends. The interior lumen 26 of the needle is in communication with the interior lumen 12 of the catheter assembly 6.

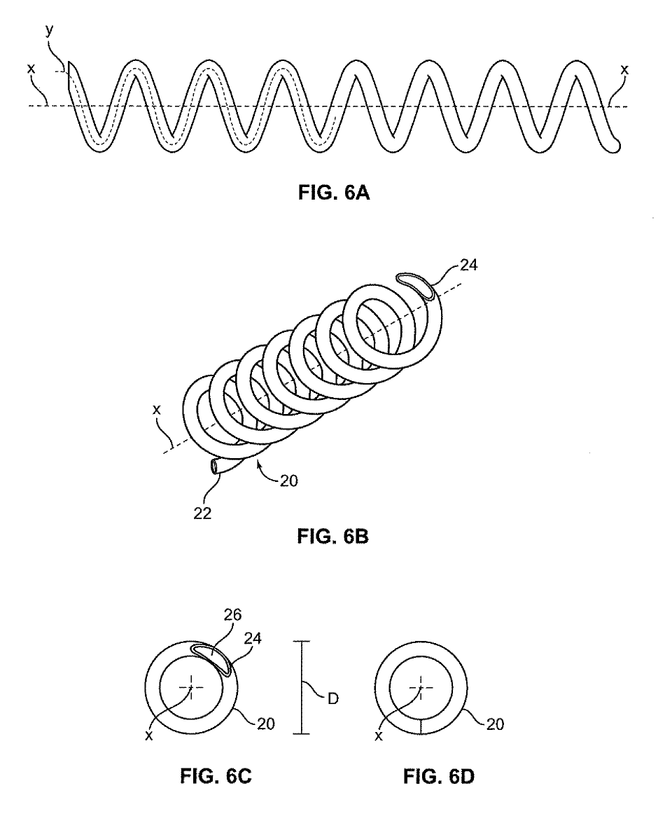

The needle 20 may be configured in any number shapes, lengths and/or sizes, such as straight, curved or any combination thereof. Preferably, the needle 20 is configured in a convoluted or serpentine shape. It is also preferable that the needle is a small gauge, such as 20 gauge or smaller diameter, so that fluid leakage is minimized when a needle disposed in the heart is removed. For example, the helical shape as seen in exemplary FIGS. 6A and 6B is such a shape. A helical needle configuration may be preferable for various reasons. In one example, a helical needle 20 may be rotated, or “screwed” into the tissue 28 as illustrated in FIGS. 2 and 3 by simply rotating the catheter assembly 6, thereby allowing ease of insertion. Also, any pressure created by fluid 30 flowing through the needle 20 would not push the needle backwards and out of the tissue 28, (which may tend to occur, for example, with a straight or linear needle), because the helical shape serves to anchor the needle 20 in the tissue 28. Further, a needle 20 having a helical shape is less likely to leak blood through the pathway 34 created in tissue 28 by the needle puncture. This is due to the fact that the pathway 34 created in the tissue 28 by a helical needle 20 is not a straight, or linear pathway. Instead, a helical needle 20 creates a longer, serpentine pathway 34 that is relatively parallel to the tissue 28 being penetrated (as compared to the shorter, linear puncture pathway that is generally perpendicular to the punctured tissue created by a straight needle). Accordingly, blood is less likely to leak through the winding, serpentine puncture pathway 34 created by a helical needle 20. Yet another advantage of a helical needle 20 is that following a needle puncture, the pathway 34 created in the tissue, having a relatively longer length and serpentine path, will become compressed by the surrounding tissue 28 upon removal of the needle 20. This not only serves to prevent bleeding through the pathway 34 from the injection site 32, but also serves to reduce bleeding at the puncture site.

Further, the needle 20 has a length L (measured along the axis of the helix “X-X”) that is sufficient to penetrate through the various chamber walls of the heart 1, as shown in FIGS. 2 and 3. In one example, the needle 20 has a length longer than the thickness of the ventricle wall. A right ventricle wall may have a thickness of approximately 10 mm or more. Accordingly, the needle 20 preferably has an effective length of between 10 mm and 20 mm, such that the needle can penetrate the thickest portion of a ventricle wall. As illustrated in FIG. 3, the helical needle 20 has a length sufficient to penetrate the wall of the right ventricle 5, even when inserted into the ventricle 5 wall at an angle.

It is preferable that the diameter of the needle 20 be as small as possible for the desired use and for the particular procedure being performed to minimize the possibility of bleeding. Selecting a needle 20 having a small diameter may serve to reduce and/or prevent bleeding in the area of penetration 34. The needle 20 may preferably be of a helical shape, as seen, for example, in FIGS. 6A and 6B. The diameter D of the needle helix may be uniform, as shown in FIG. 6A, or may vary along the length of the helix. In one example, the helical coil may have an outer diameter in the range of 0.200 to 0.050 in. using needle gauge in the range of 20-32 gauge. In another example, the diameter D of the needle helical coil is 0.100 in. The coil 20 may be made of a variety of known materials, such as metal or rigid polymer, including, but not limited to stainless steel. In one non-limiting example, the needle 20 is made of 27 gauge (0.406 mm O.D.; 0.191 mm I.D.) stainless steel hypodermic tubing.

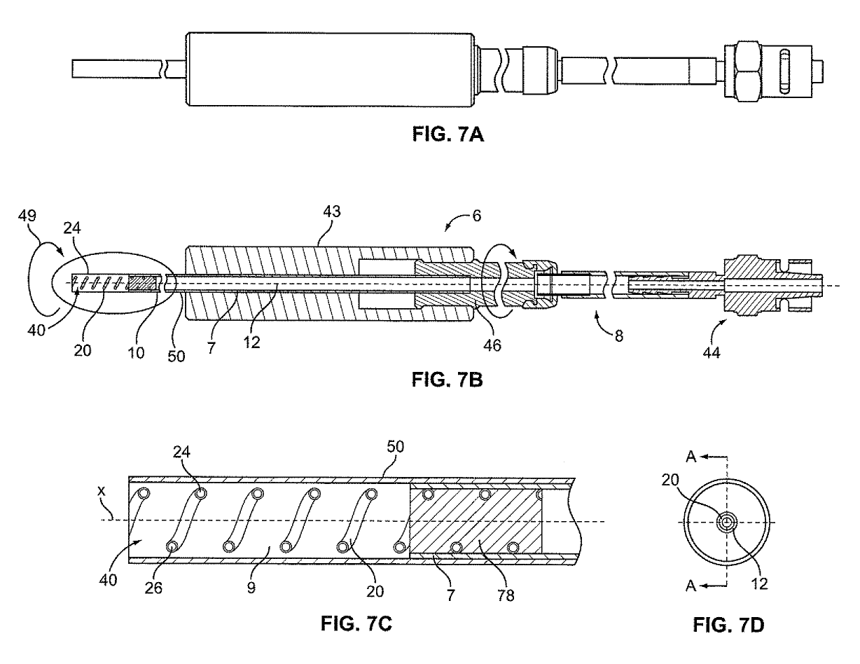

As illustrated in FIGS. 4, 7B-C and FIGS. 8A-C, the needle 20 may be axially movable. As depicted by the arrow in FIG. 4, for example, the needle may be axially movable within the lumen 9 of sheath 50. As seen in FIGS. 7A-C, the needle 20 is shown in a generally retracted position 40, such that substantially the entire length of the needle 20 is contained or housed within the lumen 9 of sheath 50. The needle 20 desirably remains in this retracted position 40 until the catheter assembly 6 has been positioned in a selected location within the heart 1. In other words, during the advancement of the catheter assembly 6 through a patient's vascular system, the needle 20 is preferably contained within the lumen 9 of sheath 50 and does not extend beyond the distal end of the sheath. The needle 20 is thereby protected by the sheath 50 to prevent inadvertent contact with tissue or damage to the needle until the distal end 10 of the catheter assembly 6 is successfully positioned in a selected area, such as, for example, near or adjacent to the apex of the right ventricle 5.

Turning back now to FIG. 2, a catheter assembly 6, having a helical needle 20 carried at the distal end 10 and covered by outer sheath 50, has been advanced through the vascular system of a patient to a location in the interior of the heart 1 and, specifically, in proximity to the apex of the right ventricle 5. Sheath 50 can be a steerable sheath, as is commonly used in cardiac catheterization procedures. The helical needle can be guided to a location in the right ventricle and affixed to the heart wall by procedures commonly known and used when securing active fixation cardiac pacing leads. As shown, for example, in FIG. 2, the distal end 10 of the catheter assembly 6 and outer sheath 50 are generally adjacent to the endocardium 42 (inner surface) of the ventricle wall 5. As further illustrated in FIGS. 2 and 3, once the catheter assembly 6 is located in the selected area, the helical needle 20 may then be advanced out of the distal end of the sheath 50 and manipulated by a physician, by various known methods.

In one non-limiting example shown in FIGS. 7A-B and 8A-B, a catheter handle, shown generally at 44 is located on the proximal end 8 of the catheter assembly body, and movably attached to a piston 46. Piston 46 is axially and rotatably movable within steering collar 43, which structure enables a physician to accurately move the needle 20, as desired. As illustrated in the embodiment illustrated in FIGS. 7B-C and FIGS. 8B-C, the needle 20 proximal end is embedded in and carried by a material, such as an epoxy, thereby creating a plug 78 around the needle proximal end. As illustrated in FIGS. 7C and 8C, the needle, mounted in epoxy plug 78, is secured in the distal portion of member 7. Moving member 7 axially, allows the needle 20 to thereby be extended out of, and retracted into the distal end of the catheter assembly 6. In an alternative embodiment illustrated in FIG. 8E, the epoxy plug 78 has a tube, or lumen 80 extending therethrough to accommodate a stylet or guide wire.

The needle 20 may also be rotated (as indicated by the arrow 49 in FIG. 8B) as it moves axially within lumen 9, as shown, for example, in FIGS. 4 and 8B. As the needle 20 is extended and rotated 49, the distal tip 24 of the needle penetrates into and advances into the tissue 28. More specifically, as illustrated in FIG. 2, the distal end 10 of the catheter assembly 6, covered by sheath 50, has been advanced into the right ventricle 5 to a desired location, and the needle 20 moved axially to extend from the lumen 9. After the needle 20 has been positioned in the apex of the right ventricle 5, the sheath 50 can be pulled back to expose the needle 20 in the ventricle, as shown in FIG. 8C, for example. During positioning of the needle, contrast media could be injected through the sheath 50, or through the needle to assist in visualization and placement of the needle. As the physician rotates the distal end 10 of the catheter assembly 6, the distal tip 24 of the needle 20 penetrates the endocardium 42, or inner wall of the ventricle 5.

As shown in FIGS. 2 and 3, the needle 20 is rotated 49 or screwed into the tissue 28, thereby advancing the needle through the endocardium 42 and into the myocardium 52 of the ventricle 5 wall. In a preferred embodiment, the needle 20 continues to be rotated 49 until it has been screwed through the entire thickness of the myocardium 52, with at least a portion of the distal tip 24 of the needle penetrating the epicardium 54 of the ventricle wall 5, as shown in FIG. 3. Rotation and advancement of the needle 20 is terminated after at least the distal end 24 of the needle has penetrated the epicardium 54 but before the distal end 24 has punctured or otherwise penetrated the pericardial tissue layer 56 surrounding the heart 1. This can be seen, for example, in FIG. 3, which illustrates the distal tip 24 of a needle 20 which has penetrated the myocardium 52, from the endocardium 42 to the epicardium 54, to reach a desired location between the epicardium 54 and the pericardial tissue layer 56. Rotation and advancement of the needle 20 is stopped when at least a portion of the needle has reached this location, before the needle tip 24 penetrates the pericardial tissue layer 56, as shown in FIG. 3.

In a preferred embodiment, substantially the entire length of the needle 20 is insulated, with the exception of the distal tip 24 of the needle which is not insulated. The needle may be covered by an insulating polymer layer 58 or sheath, or by any other known insulating means. As shown in FIGS. 4 and 5, the non-insulated distal tip 24 of the needle 20 preferably serves as or includes a sensor 60, which may be in electrical communication with an EKG monitor. For example, a separate electrical sensor 60 may be carried by the non-insulated distal tip 24 of the needle 20, or, if the needle is conductive, the tip of the needle itself may serve as the sensor. The sensor 60 may serve as an electrode to sense and record electrical signals in the cardiac tissue 28 when the needle 20 penetrates, and is being advanced into the myocardium 52. As the distal tip 24 of the needle 20 has exited the myocardium 52, the electrical signal dissipates, and the immediate indication of this position on the EKG monitor serves as an indication to an operating physician that the distal end 24 of the needle has passed through the myocardium 52, and is located in an area between the epicardium 54 and the pericardial tissue 56 layer.

This is illustrated in FIGS. 4 and 5. Electrical impulses/activity in the myocardium 52 and detected by the sensor 60 on the needle distal tip 24 travel through a conductor 64 within the catheter as shown in FIG. 5, and are displayed on EKG 62. An operating physician observing an EKG display of the recorded electrical activity is alerted to the location of the needle 20 based upon whether electrical activity is sensed by sensor 60 (i.e., the sensor 60 is located in myocardium 52) or no electrical activity is sensed (i.e., the sensor 60 is located outside of myocardium 52). When the needle exits the myocardium 52, as evidenced by the electrical signal, fluid may be advanced through the catheter and needle into the space between the pericardium 56 and epicardium 54 (outer surface of the myocardium) causing the pericardium 56 to separate from the epicardium 54 and facilitating manipulation of the pericardium 56 with reduced risk of damage to the myocardium or associated structures.

It is preferable that the needle 20 be introduced into healthy tissue 28. Accordingly, a sensor, such as the electrical sensor located on the distal end 24 of the needle 20 shown in FIGS. 4 and 5, could be used to determine whether the tissue 28 in a particular area is healthy based on the electrical signals emitted by the tissue. If an EKG 62 is recorded that indicates to an operating physician that the tissue 28 is, indeed, healthy, then a given procedure can be carried out in that location. If, however, an EKG 62 is recorded that indicates unhealthy tissue 28, the catheter assembly 6 could be repositioned until healthy tissue is located.

Alternatively, or in combination with a sensor 60, other methods may be employed. For example, as shown generally in FIGS. 1 and 3, fluid 30 contained in a fluid source 14 may be conveyed from the source, through the catheter and needle lumens 12, 26, to be injected or otherwise delivered by the needle 20 to a selected area. This fluid pressure to the needle could also be controlled with an on/off valve 76, shown in FIG. 8B. The fluid 30 may be pressurized at the fluid source 14, yet still remain contained within the needle lumen 26 during the time that the needle distal tip 24 is penetrating a ventricle 5 wall and is surrounded by myocardial tissue 52. For example, as shown in FIG. 2, during penetration of the myocardium 52 by the needle 20, the myocardial tissue contracts tightly around and exerts pressure upon the needle, including the distal tip 24, thereby providing enough resistance to “plug” or otherwise prevent the pressurized fluid 30 from flowing freely out of the needle 20. However, as illustrated in FIG. 3, when the distal tip 24 of the needle 20 has penetrated the myocardium 52 and exited the epicardium 54, fluid pressure is released and the fluid 66 flows from the distal end 24 of the needle 20 into the area 68 located between the epicardium 54 and pericardial tissue layer 56. This release of pressurized fluid (e.g. contrast agent 66) from the needle 20 serves as a visual indicator of needle position and specifically, that the distal end 24 of the needle has fully penetrated the myocardium 52 and is positioned in the area 68 between the epicardium 54 and pericardial tissue layer 56. The pressurized fluid exiting the needle tip also serves to move the pericardium away from the needle tip and prevent puncture of the pericardium.

In one embodiment, the pressurized fluid 30 may be a contrast media 66 or similar fluid agent commonly used to enhance the contrast and visibility of structures within the body such as the heart 1 and vascular system by medical imaging. As shown in FIG. 3, once the needle 20 has penetrated the myocardium 52, the contrast fluid 66 flowing into the area 68 between the epicardium 54 and pericardial tissue layer 56 separates the two tissue layers and may be observed flowing into the area 68 using fluoroscope or other commonly used imaging techniques.

As further illustrated in FIG. 3, once the epicardium 54 has been penetrated, the distal end 24 of the needle 20 resides in the area 68 located between the epicardium 54 and pericardial tissue layer 56. Contrast fluid 66 flows from the source 14, through the catheter and needle lumens 12, 26, and is injected or otherwise delivered into the area 68. As fluid 30 continuously flows to and collects between the epicardium 54 and pericardium 56, sufficient pressure is created such that the pericardial tissue layer 56 is separated from the epicardium 54. Where the fluid 30 being injected is fluid contrast agent 66, the separation of the pericardial layer 56 can be observed by the physician by the variety of known methods described above.

By observing the separation of the pericardium 56 from the epicardium 54, the physician can more accurately control the rate of fluid flow to the area, thereby controlling the rate of separation of the tissue, as well as control the distance that the pericardium 56 is separated from the epicardium 54 and the space created by the fluid 30 between the tissue layers. Once separation of the pericardial tissue 56 has been satisfactorily achieved by the injection of contrast fluid 66, a physician can clearly observe the location of the separated tissue relative to the rest of the heart 1, as well as the size of the area 68 created between the tissue layers by the injected fluid 66, as by a fluorescence.

Turning now to FIG. 5, the inner tubular member 7, located within sheath 50 (of FIG. 4), is illustrated with the sheath removed. This member itself may also be referred to as a catheter, having a length, a proximal end and a distal end and a lumen 12 extending therethrough. FIG. 5 illustrates a further feature, specifically, a fluid connector 70 may be located at the proximal end 8 of the catheter assembly 6. The connector, illustrated as a T-connector 70 in FIG. 5, is preferably attached to a pressure monitor 72. The pressure monitor 72 is adapted to detect and monitor the flow rate and pressure of fluid 66 injected through the catheter and needle lumens 12, 26. In a preferred embodiment, the pressure monitor 72 continuously detects the pressure created by the injection of fluid 30, such as contrast fluid 66, into the area 68 located between the epicardium 54 and the pericardial tissue layer 56. The pressure monitor 72 is adapted to provide feedback to an operating physician regarding the detected fluid pressure. Such feedback may be provided to a physician in a variety of known methods, for example, on a display monitor, gauge, or the like associated with the pressure monitor 72. The blood pressure of the patient, which is also being monitored for the duration of any given procedure, may be displayed with the fluid pressure as an additional source of informational feedback that is provided to the physician, along with fluid pressure detected by the pressure monitor 72, which may be indicative of any tamponade effect of the fluid.

As also seen in FIG. 5, the pressure monitor 72 may also be combined with a controller 74 for generating a signal to a physician when the pressure of fluid injected into the area 68 between the epicardium 54 and pericardial tissue layer 56 exceeds a certain level. The controller 74 may also be adapted to compare the blood pressure of the patient with the fluid pressure detected by the pressure monitor 72. Typically, the fluid pressure created between the epicardium 54 and the pericardial layer 56 mirrors the blood pressure of the patient and more specifically, the fluid pressure may be presented as a percentage (%) of a patient's blood pressure, and the physician may choose to limit the fluid pressure to below a certain percentage of the patient's blood pressure.

One reason for this is that excessive fluid pressure upon the epicardium 54 may lead to one or more unsafe or even emergency conditions for a patient and may compromise the normal heart rhythm. For example, cardiac tamponade, also known as pericardial tamponade, is a condition that occurs when fluid rapidly accumulates in the area 68 between the epicardium 54 and pericardial tissue layer 56. If the accumulated fluid 30 significantly elevates pressure on the heart 1, it may prevent one or more of the ventricles from properly filling. This may result in the heart 1 ineffectively pumping blood, leading to shock and, if not addressed, even death. Thus, when the patient's cardiac output is impacted by excessive intrapericardial pressure, the controller 74 would indicate this condition, and provide a warning signal to the physician to reduce or even terminate the flow of fluid 30 to the area 68 between the epicardium 54 and pericardium 56. The controller could also be associated with a flow control valve to reduce or stop flow through the catheter, or potentially withdraw fluid from the pericardium.

The separation of the pericardial tissue 56 layer from the epicardium 54 has several advantages. The primary advantage is that it allows penetration of the pericardium with a separate medical instrument, while reducing the likelihood of penetrating the heart wall. For example, it creates an unobstructed working space or area 68 for a physician to access the epicardium 54 during a given medical procedure. A physician can more easily access, and then manipulate medical and/or diagnostic instruments in the space created between the epicardium 54 and pericardial tissue 56 layer because the pericardium 56, which typically fits tightly around the epicardium 54 in its natural state, has been moved away from the epicardial surface. This further creates an unobstructed and clearer view of the epicardium 54 for an operating physician performing a medical procedure on or around the heart.

In one non-limiting example, a medical procedure requiring ablation of cardiac tissue may involve a surgical site, i.e., a site for ablation of cardiac tissue to treat atrial fibrillation, located on or near the epicardium 54. The creation of an unobstructed working space by separating the pericardial tissue layer 56 from the epicardium 54 allows a physician to more easily access the epicardium 54 and manipulate medical instruments in the area with greater accuracy and precision. A given procedure can thereby be performed with a reduced risk of potentially damaging healthy cardiac tissue or tissue surrounding the surgical site that may otherwise occur when closeness of the pericardial tissue layer 56 interferes with and restricts access to the epicardium 54.

Separating the pericardium 56 from the epicardium 54 to create unobstructed access to the epicardium may have a variety of other advantages not described here in detail, but which may be understood by one of skill in the art, including, but not limited to, providing access to a physician requiring access to the epicardium by one or more of intercostal access and subxyphoid access approaches.

In one non-limiting example, exemplary methods of separating pericardial tissue 56 from the epicardium 54 of the heart utilizing the above-described apparatus 2 may first include inserting a guide wire into the interior of the right ventricle of the heart. A catheter, having a proximal end, a distal end, a guide wire lumen 80 and a fluid lumen extending therebetween is advanced over the guide wire, into the right ventricle. The method further includes advancing helical needle 20 out of the distal end of the catheter assembly 6 while preferably retracting a sheath 50 which covers the catheter body, to expose helical needle 20 within the right ventricle. Preferably, the method includes penetrating the endocardium 42, or inner surface of the heart, with needle 20, by rotating the needle and further advancing the needle into the myocardium while fluid pressure is exerted through the needle until a distal end 24 of the needle 20 extends beyond the epicardium, but does not penetrate pericardial tissue 56. The claimed method may further include providing a sensor on the distal end of the needle 20 to sense electrical signals in the myocardium to be displayed on an associated EKG monitor, thereby providing information to an operating physician of the relative location of the needle based on the sensed electrical signals.

When the sensor on the tip of the needle exits the myocardium, the EKG signal drops off, indicating that the needle has pierced through the epicardium 54. Contrast fluid from a source 14 may then be delivered through the catheter and needle lumens 12, 26 to the area 68 between the epicardium and pericardium, at a pressure sufficient to cause separation of the pericardium 56 from the epicardium 54 of the heart. Contrast media flowing into the area 68 between the epicardium and pericardium may be directly observed, via fluoroscope, flowing into the area. Alternatively, no sensor may be employed and pressure is applied to the contrast fluid as the needle 20 is advanced through the myocardium 52. As soon as the needle exits the myocardium, contrast agent will flow into the space between the epicardium 54 and pericardium 56 and be visible to the surgeon. In yet another embodiment, the claimed method may detect and monitor, and/or control flow rate and pressure of fluid injected through the catheter and needle lumens 12, 26, into the area 68 between the epicardium 54 and pericardium 56.

Following from the above description and exemplary embodiments, it should be apparent to those of ordinary skill in the art that, while the foregoing constitute exemplary embodiments of the present disclosure, the disclosure is not necessarily limited to these precise embodiments and that changes may be made to these embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the disclosure discussed herein in order to fall within the scope of any claims, since the invention is defined by the claims and since inherent and/or unforeseen advantages of the present disclosure may exist even though they may not have been explicitly discussed herein.

What is claimed is:

1. Apparatus for separating pericardial tissue from the epicardium of the heart of a patient comprising:
a catheter comprising an elongated body including a proximal end and a distal end and a lumen extending between the proximal and distal ends,
a helical needle carried at the distal end of the catheter, said needle including a proximal end, a distal end and a lumen extending between said needle proximal and distal ends and in fluid communication with said catheter lumen, said needle being of a length sufficient to penetrate myocardial tissue from the endocardium to and through the epicardium,
said catheter including a coupling for communication with a fluid source for flow of fluid through said catheter lumen and said needle lumen to a location between the epicardium and pericardial tissue to separate the pericardial tissue from the epicardium of the heart.

2. The apparatus of claim 1 further comprising:
the needle having an insulative cover so that the distal end is electrically conductive, the distal end comprising an electrical sensor; and an EKG connected to the sensor.

3. The apparatus of claim 1 further comprising a pressure monitor for detecting and monitoring fluid pressure through the lumens and a controller for generating a signal based on the measured fluid pressure.

4. The apparatus of claim 3 wherein the controller compares the patient's blood pressure to the fluid pressure through the lumens.

5. A method of accessing the pericardial space between the epicardium and the pericardium of the heart of a patient comprising:
providing a catheter with a distal end and having a fluid lumen, the distal end comprising a hollow helical needle in communication with the fluid lumen and having an operative length sufficient to penetrate through a chamber wall of the heart;
providing a source of pressurized fluid in communication with the lumen of the catheter;
introducing the distal end of the catheter into a chamber of the heart;
contracting the chamber wall with the helical needle;
introducing pressurized fluid into the catheter lumen;
advancing the helical needle through the chamber wall until the hollow needle penetrates the wall and releases pressurized fluid into the pericardial space; and
releasing sufficient pressurized fluid into the pericardial space to separate the pericardium from the epicardium.

6. The method of claim 5 further comprising providing the pressurized fluid with a contrast agent visible using medical imaging techniques and viewing the contrast agent in the pressurized fluid released in to the pericardial space to confirm the penetration of the chamber wall by the helical needle and to determine the size the pericardial space.

7. The method of claim 5 further comprising:
the helical needle having a distal tip and providing the distal tip with an electrical sensor;
providing an EKG monitor connected to the sensor;
measuring the EKG as the helical needle advances through the chamber wall; and
detecting the penetration of the distal tip of the helical needle through the chamber wall by the change in the EKG.

8. The method of claim 5 further comprising:
providing a pressure monitor for detecting the pressure of the fluid within the catheter; and
providing a controller for generating a signal when the fluid pressure exceeds a predetermined level.

9. The method of claim 8 wherein the controller compares the pressure of the fluid within the catheter to the patient's blood pressure, and provides a signal when the pressure within the catheter exceeds the patient's blood pressure.
